(19) United States
(12) Patent Application Publication
(10) Pub. No.: US 2026/0043781 A1
(43) Pub. Date: Feb. 12, 2026

(54) ON-DEMAND DETERMINATION OF FECAL COLIFORM PRESENCE IN WATER RESOURCES

(71) Applicant: RAMOT AT TEL-AVIV UNIVERSITY LTD., Tel Aviv (IL)

(72) Inventors: Hadas MAMANE, Tel Aviv (IL); Asaf PRAS, Tel Aviv (IL)

(21) Appl. No.: 18/998,778

(22) PCT Filed: Jul. 26, 2023
(86) PCT No.: PCT/IL2023/050778

§ 371 Date: Jan. 27, 2025

Related U.S. Application Data
(60) Provisional application No. 63/392,288, filed on Jul. 26, 2022.

(51) Int. Cl. G01N 33/18; G01N 21/53; G01N 27/07; G01N 27/416; G01N 35/00; G16C 20/70

(52) U.S. Cl. CPC G01N 33/1826; G01N 21/53; G01N 27/07; G01N 27/4167; G01N 33/182; G01N 35/00871; G16C 20/70; G01N 2035/0091

(57) ABSTRACT

There is provided a water quality probe system, the system comprising: one or more water contact sensors; a geolocation unit; a processing circuitry (PC), operably connected to the one or more water contact sensors and to the geolocation unit, the PC being configurable to: receive, from one or more of the water contact sensors, data indicative of one or more water characteristics sensed from contacted water; receive, from the geolocation unit, data indicative of a current geographical location; and determine data indicative of whether the contacted water satisfies one or more water quality criteria, utilizing, at least, the received sensed water characteristics and the current geographical location.

### TECHNICAL FIELD

The presently disclosed subject matter relates to probing and evaluating water resources, and in particular to real-time detection of water resource contamination.

### BACKGROUND

Problems of implementation in systems of water resource evaluation have been recognized in the conventional art and various techniques have been developed to provide solutions.

### GENERAL DESCRIPTION

According to one aspect of the presently disclosed subject matter there is provided a water quality probe system, the system comprising:
a) one or more water contact sensors;
b) a geolocation unit;
c) a processing circuitry (PC), operably connected to the one or more water contact sensors and to the geolocation unit, the PC being configurable to:
   a. receive, from one or more of the water contact sensors, data indicative of one or more water characteristics sensed from contacted water;
   b. receive, from the geolocation unit, data indicative of a current geographical location; and
   c. determine data indicative of whether the contacted water satisfies one or more water quality criteria, utilizing, at least, the received sensed water characteristics and the current geographical location.
      In addition to the above features, the system according to this aspect of the presently disclosed subject matter can comprise one or more of features (i) to (xiii) listed below, in any desired combination or permutation which is technically possible:
(i) at least one of the one or more water contact sensors is selected from of a list consisting of:
   a. a pH sensor,
   b. a temperature sensor,
   c. an electroconductivity sensor,
   d. a turbidity sensor,
   e. a dissolved oxygen sensor, and
   f. a total dissolved solids sensor
(ii) the geolocation unit is a global positioning system (GPS).
(iii) the system is handheld
(iv) the PC is configured to perform a.-c.
(v) the PC is further configured to perform the determining by utilizing, at least, a trained machine learning model
(vi) additionally comprising a communications link, and the PC is further configured to perform the determining by receiving, from a remote server, data indicative of the whether the contacted water satisfies one or more water quality criteria,
the receiving from the remote server being at least partially responsive to the PC transmitting, at least, data derivative of at least part of the received sensed data and data derivative of the current geographical location to the remote server.
(vii) the PC is further configured to: present, on a user interface, an indication of whether the contacted water satisfies at least one criterion of the one or more water quality criteria.
(viii) the one or more water sensors comprises:
   a. a pH sensor,
   b. a temperature sensor,
   c. an electroconductivity sensor,
   d. a turbidity sensor, and
   e. a dissolved oxygen sensor; and
the PC utilizes, as inputs to the trained machine learning model, at least:
   a measured pH of the water source,
   a temperature of the water source,
   an electroconductivity of the water source,
   a turbidity of the water source, and
   a measured dissolved oxygen level of the water source; and
at least one of the one or more water quality criteria determined by the trained machine learning model is whether a concentration of fecal coliforms in the contacted water lies between a given lower bound and a given upper bound.
(ix) the one or more water sensors further comprises:
a total dissolved solids sensor, and
the PC further utilizes a measured total dissolved solids of the water source as an input to the trained machine learning model.
(x) the one or more water sensors comprises:
   a. a pH sensor,
   b. a temperature sensor,
   c. an electroconductivity sensor,
   d. a turbidity sensor, and
   e. a dissolved oxygen sensor; and
the PC receives, at least, from the one or more water sensors, data indicative of:
   a measured pH of the water source,
   a temperature of the water source,
   an electroconductivity of the water source,
   a turbidity of the water source, and
   a measured dissolved oxygen level of the water source; and
at least one of the one or more water quality criteria is whether a concentration of fecal coliforms in the contacted water lies between a given lower bound and a given upper bound.
(xi) the one or more water sensors further comprises:
a total dissolved solids sensor, and
the PC further receives data indicative of a measured total dissolved solids of the water source.
(xii) the one or more water contact sensors comprises:
   a. a pH sensor,
   b. a temperature sensor, and
   c. an electroconductivity sensor; and
the PC utilizes, as inputs to the trained machine learning model, at least:
   a. a measured pH of the water source,
   b. a temperature of the water source,
   c. an electroconductivity of the water source; and
one of the one or more water quality criteria determined by the trained machine learning model is whether fluoride content of the contacted water meets a fluoride content threshold.
(xiii) the one or more water contact sensors comprises:
   a. a pH sensor,
   b. a temperature sensor, and
   c. an electroconductivity sensor; and
the PC receives, at least, from the one or more water sensors, data indicative of:
   a. a measured pH of the water source,
   b. a temperature of the water source,
   c. an electroconductivity of the water source; and
one of the one or more water quality criteria received from the remote server is whether fluoride content of the contacted water meets a fluoride content threshold.

According to another aspect of the presently disclosed subject matter there is provided a computer-implemented method of determining whether contacted water satisfies one or more water quality criteria, the method comprising:
a) receiving, from one or more water contact sensors, data indicative of one or more water characteristics sensed from the contacted water;
b) receiving, from a geolocation unit, data indicative of a current geographical location; and
c) determining data indicative of whether the contacted water satisfies one or more water quality criteria, utilizing, at least, the received sensed water characteristics and the current geographical location.

This aspect of the disclosed subject matter can further optionally comprise one or more of features (i) to (xiii) listed above with respect to the system, mutatis mutandis, in any desired combination or permutation which is technically possible.

According to another aspect of the presently disclosed subject matter there is provided a computer program product comprising a computer readable non-transitory storage medium containing program instructions, which program instructions when read by a processor, cause the processing circuitry to perform a method of determining whether contacted water satisfies one or more water quality criteria, the method comprising:
a) receiving, from one or more water contact sensors, data indicative of one or more water characteristics sensed from the contacted water;
b) receiving, from a geolocation unit, data indicative of a current geographical location; and
c) determining data indicative of whether the contacted water satisfies one or more water quality criteria, utilizing, at least, the received sensed water characteristics and the current geographical location.

This aspect of the disclosed subject matter can further optionally comprise one or more of features (i) to (xiii) listed above with respect to the system, mutatis mutandis, in any desired combination or permutation which is technically possible.

According to one aspect of the presently disclosed subject matter there is provided a system of determining whether a concentration of fecal coliforms in contacted water of a water source meets a fecal coliform concentration criterion, the system comprising a processing circuitry (PC) configured to:
a) receive, at least, data indicative of:
   a geographic location of the water source,
   a pH of the contacted water,
   a temperature of the contacted water,
   an electroconductivity of the contacted water,
   a turbidity of the contacted water, and
   a dissolved oxygen level of the contacted water; and
b) utilize, at least, the received data in conjunction with a machine learning model trained to determine data indicative of whether a concentration of fecal coliforms in contacted water of a water source meets the fecal coliform concentration criterion,
   wherein the machine learning model was trained utilizing, at least, a set of training samples, wherein each training sample comprises:
      data indicative of a geographic location of an origin of a respective water sample;
      data indicative of a measured pH of the respective water sample,
      data indicative of a temperature of the respective water sample,
      data indicative of an electroconductivity of the respective water sample,
      data indicative of a turbidity of the contacted water,
      data indicative of a dissolved oxygen level of the contacted water, and
      ground truth data indicative of whether a concentration of fecal coliforms in the sample meets the fecal coliform concentration criterion.
         In addition to the above features, the system according to this aspect of the presently disclosed subject matter can comprise one or more of features (i) to (iv) listed below, in any desired combination or permutation which is technically possible:
(i) wherein the fecal coliform concentration criterion is whether the fecal coliform concentration lies between a given lower bound and a given upper bound.
(ii) the geographic location comprises an identifier of a geographical region.
(iii) the geographic location comprises a longitude and a latitude.
(iv) the received data further comprises:
   data indicative of an origin type of the water source;
and each training sample further comprises:
   data indicative of an origin type of the respective water sample.

According to another aspect of the presently disclosed subject matter there is provided a computer-implemented method of determining whether a concentration of fecal coliforms in contacted water of a water source meets a fecal coliform concentration criterion, the method comprising:
a) receiving, at least, data indicative of:
   a geographic location of the water source,
   a pH of the contacted water,
   a temperature of the contacted water,
   an electroconductivity of the contacted water,
   a turbidity of the contacted water, and
   a dissolved oxygen level of the contacted water; and
b) utilizing, at least, the received data in conjunction with a machine learning model trained to determine data indicative of whether a concentration of fecal coliforms in contacted water of a water source meets the fecal coliform concentration criterion, wherein the machine learning model was trained utilizing, at least, a set of training samples, wherein each training sample comprises:
   data indicative of a geographic location of an origin of a respective water sample;
   data indicative of a measured pH of the respective water sample,
   data indicative of a temperature of the respective water sample,
   data indicative of an electroconductivity of the respective water sample,
   data indicative of a turbidity of the contacted water,
   data indicative of a dissolved oxygen level of the contacted water, and
   ground truth data indicative of whether a concentration of fecal coliforms in the sample meets the fecal coliform concentration criterion.

This aspect of the disclosed subject matter can further optionally comprise one or more of features (i) to (iv) listed above with respect to the system, mutatis mutandis, in any desired combination or permutation which is technically possible.

According to another aspect of the presently disclosed subject matter there is provided a computer program product comprising a computer readable non-transitory storage medium containing program instructions, which program instructions when read by processor, cause the processing circuitry to perform a method of determining whether a concentration of fecal coliforms in contacted water of a water source meets a fecal coliform concentration criterion, the method comprising:
a) receiving, at least, data indicative of:
   a geographic location of the water source,
   a pH of the contacted water,
   a temperature of the contacted water,
   an electroconductivity of the contacted water,
   a turbidity of the contacted water, and
   a dissolved oxygen level of the contacted water; and
b) utilizing, at least, the received data in conjunction with a machine learning model trained to determine data indicative of whether a concentration of fecal coliforms in contacted water of a water source meets the fecal coliform concentration criterion, wherein the machine learning model was trained utilizing, at least, a set of training samples, wherein each training sample comprises:
   data indicative of a geographic location of an origin of a respective water sample;
   data indicative of a measured pH of the respective water sample,
   data indicative of a temperature of the respective water sample,
   data indicative of an electroconductivity of the respective water sample,
   data indicative of a turbidity of the contacted water,
   data indicative of a dissolved oxygen level of the contacted water, and
   ground truth data indicative of whether a concentration of fecal coliforms in the sample meets the fecal coliform concentration criterion.

This aspect of the disclosed subject matter can further optionally comprise one or more of features (i) to (iv) listed above with respect to the system, mutatis mutandis, in any desired combination or permutation which is technically possible.

According to one aspect of the presently disclosed subject matter there is provided system of determining whether fluoride content of contacted water of a water source meets a fluoride content criterion, the system comprising a processing circuitry (PC) configured to:
c) receive, at least, data indicative of:
   a geographic location of the water source,
   a pH of the contacted water,
   a temperature of the contacted water,
   an electroconductivity of the contacted water; and
d) utilize, at least, the received data in conjunction with a machine learning model trained to determine data indicative of whether a fluoride content of contacted water of a water source meets the fluoride content criterion,
   wherein the machine learning model was trained utilizing, at least, a set of training samples, wherein each training sample comprises:
      data indicative of a geographic location of an origin of a respective water sample;
      data indicative of a measured pH of the respective water sample,
      data indicative of a temperature of the respective water sample,
      data indicative of an electroconductivity of the respective water sample, and
      ground truth data indicative of whether a fluoride content s in the respective sample meets the fluoride content criterion.
         In addition to the above features, the system according to this aspect of the presently disclosed subject matter can comprise one or more of features (i) to (iv) listed below, in any desired combination or permutation which is technically possible:
(v) wherein the fluoride content criterion is whether fluoride content lies between a given lower bound and a given upper bound.
(vi) the geographic location comprises an identifier of a geographical region.
(vii) the geographic location comprises a longitude and a latitude.
(viii) the received data further comprises:
   data indicative of an origin type of the water source;
and each training sample further comprises:
   data indicative of an origin type of the respective water sample.

According to another aspect of the presently disclosed subject matter there is provided a computer-implemented method of determining whether fluoride content of contacted water of a water source meets a fluoride content criterion, the method comprising:
c) receiving, at least, data indicative of:
   a geographic location of the water source,
   a pH of the contacted water,
   a temperature of the contacted water,
   an electroconductivity of the contacted water; and
d) utilizing, at least, the received data in conjunction with a machine learning model trained to determine data indicative of whether a fluoride content of contacted water of a water source meets the fluoride content criterion, wherein the machine learning model was trained utilizing, at least, a set of training samples, wherein each training sample comprises:
   data indicative of a geographic location of an origin of a respective water sample;
   data indicative of a measured pH of the respective water sample,
   data indicative of a temperature of the respective water sample,
   data indicative of an electroconductivity of the respective water sample, and
   ground truth data indicative of whether a fluoride content of the respective water sample meets the fluoride content criterion.

This aspect of the disclosed subject matter can further optionally comprise one or more of features (i) to (iv) listed above with respect to the system, mutatis mutandis, in any desired combination or permutation which is technically possible.

According to another aspect of the presently disclosed subject matter there is provided a computer program product comprising a computer readable non-transitory storage medium containing program instructions, which program instructions when read by a processor, cause the processing circuitry to perform a method of determining whether fluoride content of contacted water of a water source meets a fluoride content criterion, the method comprising:
c) receiving, at least, data indicative of:
   a geographic location of the water source,
   a pH of the contacted water,
   a temperature of the contacted water,
   an electroconductivity of the contacted water; and
d) utilizing, at least, the received data in conjunction with a machine learning model trained to determine data indicative of whether fluoride content of contacted water of a water source meets a fluoride content criterion, wherein the machine learning model was trained utilizing, at least, a set of training samples, wherein each training sample comprises:
   data indicative of a geographic location of an origin of a respective water sample;
   data indicative of a measured pH of the respective water sample,
   data indicative of a temperature of the respective water sample,
   data indicative of an electroconductivity of the respective water sample, and
   ground truth data indicative of whether fluoride content of the respective water sample meets a fluoride content criterion.

This aspect of the disclosed subject matter can further optionally comprise one or more of features (i) to (iv) listed above with respect to the system, mutatis mutandis, in any desired combination or permutation which is technically possible.

### BRIEF DESCRIPTION OF THE DRAWINGS

In order to understand the invention and to see how it can be carried out in practice, embodiments will be described, by way of non-limiting examples, with reference to the accompanying drawings, in which:

FIG. 1A illustrates an example machine learning model usable as a component of a water quality probe system for determination of water quality, in accordance with some embodiments of the presently disclosed subject matter;

FIG. 1B illustrates an example water probe system for determination of water quality, in accordance with some embodiments of the presently disclosed subject matter;

FIGS. 2A-2D are block diagrams of example water probe systems for determination of water quality, in accordance with some embodiments of the presently disclosed subject matter; and

FIG. 3 is a flow diagram of an example method of determining whether contacted water meets a particular water quality criterion, in accordance with some embodiments of the presently disclosed subject matter; and

FIG. 4 is a flow diagram of another example method of determining whether contacted water meets a particular water quality criterion, in accordance with some embodiments of the presently disclosed subject matter.

### DETAILED DESCRIPTION

In the following detailed description, numerous specific details are set forth in order to provide a thorough understanding of the invention. However, it will be understood by those skilled in the art that the presently disclosed subject matter may be practiced without these specific details. In other instances, well-known methods, procedures, components and circuits have not been described in detail so as not to obscure the presently disclosed subject matter.

Unless specifically stated otherwise, as apparent from the following discussions, it is appreciated that throughout the specification discussions utilizing terms such as “processing”, “computing”, “comparing”, “determining”, “calculating”, “receiving”, “providing”, “obtaining”, “estimating” or the like, refer to the action(s) and/or process(es) of a computer that manipulate and/or transform data into other data, said data represented as physical, such as electronic, quantities and/or said data representing the physical objects. The term “computer” should be expansively construed to cover any kind of hardware-based electronic device with data processing capabilities including, by way of non-limiting example, the processor, mitigation unit, and inspection unit therein disclosed in the present application.

The terms “non-transitory memory” and “non-transitory storage medium” used herein should be expansively construed to cover any volatile or non-volatile computer memory suitable to the presently disclosed subject matter.

The operations in accordance with the teachings herein may be performed by a computer specially constructed for the desired purposes or by a general-purpose computer specially configured for the desired purpose by a computer program stored in a non-transitory computer-readable storage medium.

Embodiments of the presently disclosed subject matter are not described with reference to any particular programming language. It will be appreciated that a variety of programming languages may be used to implement the teachings of the presently disclosed subject matter as described herein.

According to the World Health Organization (WHO), at least 2 billion people were using drinking water sources that were contaminated with microbial contamination. Moreover, by 2025, half of the world's population will be living in water-stressed areas. Surface water bodies are particularly vulnerable to microbial contamination from natural and human activities, including leaching of animal manure, leaking septic tanks, wastewater for irrigation, raw sewage discharge and stormwater runoff. Drinking untreated contaminated water may result in waterborne diseases, and has been implicated in the daily death rate of more than 800 children under the age of 5 years from diarrheal diseases due to poor sanitation, poor hygiene, and unsafe drinking water.

Inadequate management of wastewater results in polluted drinking water and puts public in danger. Thus, detection of microbial contamination is necessary to develop protective risk management. Therefore, water quality should be routinely analyzed, especially in areas with higher risk of sewage contamination and with less advanced or efficient water-treatment facilities. The analysis of fecal coliforms (FC) also referred as thermotolerant coliform, in water, performed in most cases by the Escherichia coliform (E. coli) test or FC test, is tedious as it involves sampling, transporting the samples to the laboratory, and analyzing and reporting the results, all of which can take more than 24 h. Additional FC-detection methods include electrochemical biosensors and optical sensors. However, most of these methods either do not provide real-time results, or are expensive, require trained operators and are not suited for in-situ measurements, and hence cannot be used as an affordable and easy to operate solution in large scale.

Timely and accurately assessing fecal coliform levels in water is essential for effective decision-making, proactive risk management, and safeguarding human well-being. Therefore, the World Health Organization (WHO) has established guidelines for FC levels in drinking water to protect public health. According to these guidelines, the maximum allowable concentration of FC in drinking water should not exceed 0 colony-forming units (CFUs) per 100 milliliters of water for microbial safety (WHO, 2017). In addition, the WHO has grouped fecal contamination levels into five risk categories: very low risk (0 CFU/100 ml), low risk (1-9 CFU/100 ml), intermediate risk (10-99 CFU/100 ml), high risk (100-999 CFU/100 ml), and very high risk (≥1000 CFU/100 ml) (WHO, 1997). Studies show that the association between FC levels according to these categories in drinking water and waterborne diseases matches a dose-response effect. For the low-risk category, there is limited evidence of increased odds of waterborne diseases, such as diarrhea, and in contrast, multiple studies show that high-risk spikes of fecal contamination carry more risk to human health. High-risk spikes of fecal contamination are associated with heavy defecation or drainage of heavy rainfall directly into source waters or washing of contaminated items like diapers. Therefore, these contamination incidents'size and frequency can be variable and will be missed by traditional microbial water testing.

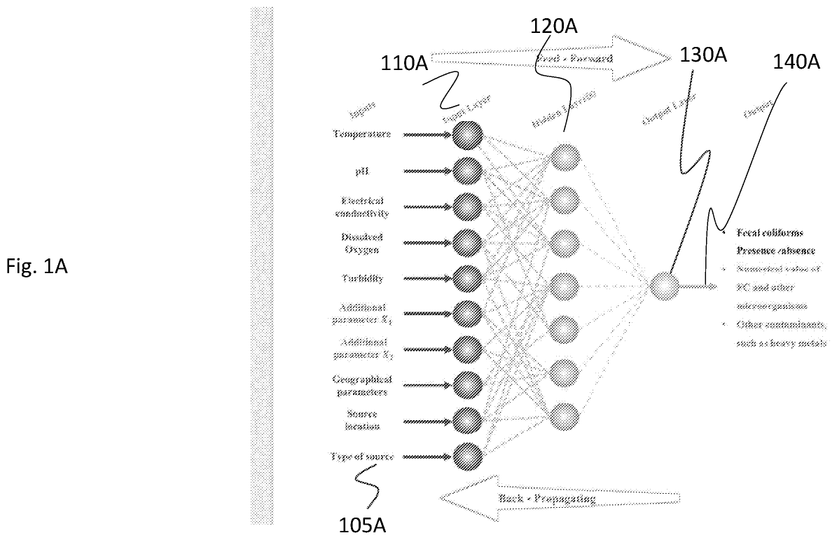

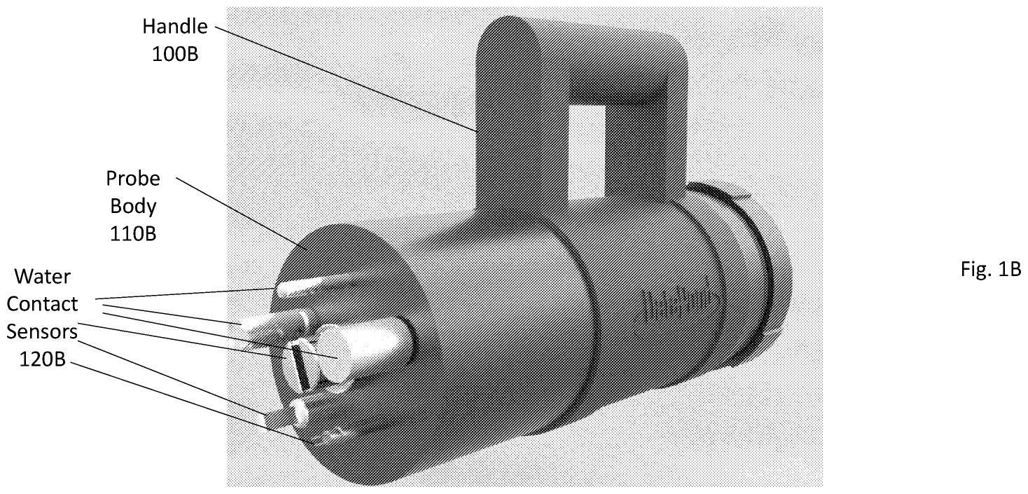

FIG. 1A illustrates an example machine learning model usable as a component of a water quality probe system for determination of water quality, in accordance with some embodiments of the presently disclosed subject matter.

Some embodiments of the presently disclosed subject matter utilize a machine learning model to map sensed characteristics of contacted water (e.g. sources of drinking water) into estimations of whether the contacted water meets—for example—a particular water quality criterion. In some such embodiments, a multilayer perceptron—artificial neural network (MLP-ANN) is utilized. In other embodiments, a different machine learning scheme is utilized.

As shown in FIG. 1A, the ANN includes an input layer 110A, where the input variables 105A are fed into the algorithm, hidden layers 120A where the inputs are combined and processed, and an output layer 130A which produces the output 140A probability estimate.

In some embodiments of the presently disclosed subject matter, the following data can be received as inputs:
a) a pH value,
b) a temperature,
c) an electroconductivity value,
d) a turbidity value,
e) (optionally) a total dissolved solids (TDS) value, and
f) a dissolved oxygen value

In some embodiments of the presently disclosed subject matter, the ANN outputs data indicative of a whether the contacted water meets one or more water quality criteria pertaining to presence of fecal coliforms in the contacted water. By way of non-limiting example, the following can be ANN outputs:
a) a binary indication of presence/absence of fecal coliforms in the water source
b) an estimated probability of presence of fecal coliforms in the water source
c) a binary indication of presence of fecal coliforms in the water source above a certain threshold, or within a certain range (delineated by a lower bound concentration and a higher bound concentration as in the risk levels detailed above).
d) an estimated probability of concentration of fecal coliforms in the water source above a certain threshold, or within a certain range (delineated by a lower bound concentration and a higher bound concentration).
e) an estimated concentration value (e.g., in ppm, mg/L etc.) of fecal coliforms in the water source In some examples, the physical and chemical water-quality parameters that significantly impact microbial organisms'growth and survival in raw water sources include temperature, pH, turbidity, and electrical conductivity. Dissolved solids and dissolved oxygen can be associated with human and animal sources of water contamination which can lead to the presence of fecal coliforms.

In some embodiments, a feed-forward, error back-propagating MLP (BP-MLP) ANN architecture is utilized. During training, back-propagation can be used to fit the model, where the information is transited forward to the output layer, and errors are back-propagated.

In some embodiments of this architecture, during the feed-forward transition, the input layer(s)' values or features is processed into the hidden layer(s) and then processed again into the output layer. Every node in a layer affects the nodes in the subsequent layer(s). The output layer provides a probability of the sample being classified into an event occurred class, in this case being e.g. whether the contacted water meets a water quality criterion.

In some embodiments, the probability is then compared with a given threshold for classification. If the output prediction does not meet the expected outcome, it is returned to the back-propagating process as an error. In the backpropagation, weight and threshold values of the network are adjusted to tune the model to approach the expected label. This process is done for small batches of the training data until the model is trained on an entire training dataset, and then a validation dataset is used to evaluate the model. The model training and validation procedure is repeated hundreds of times to get to the best accuracy.

In some embodiments, a four-hidden-layer architecture is utilized, with each layer containing sixty nodes. A rectified linear unit (Aggarwal, 2018; Nair and Hinton, 2010) activation function can be used after each node, while at the output layer a sigmoid function (Aggarwal, 2018) can be used to transform the last layer result into a probability as [0,1]. In some embodiments, the adaptive moment estimation (ADAM) optimizer (Aggarwal, 2018; Kingma and Ba, 2014) can be utilized as a gradient descent optimizer.

In some embodiments, additional inputs are utilized:
a) geographic location
   Fecal coliforms can be associated with sources of human and animal waste e.g. human settlements or areas of animal activity. Accordingly, geographic location can be provided as an input to the machine learning model (e.g. as longitude and latitude, as a character string or numeric value indicating a particular geographic district, or in some other manner)
b) water source type
   Water source type (e.g. river, well, lake) can be utilized (e.g. provided as a character string or a number value) an input to the machine learning model
      Fluoridation of water sources is also correlated with geographic location and/or water source type (due to presence of fluoride in the earth at different locations).

Accordingly, in some embodiments of the presently disclosed subject matter, the following data is received as inputs:
a) a pH,
b) a temperature,
c) an electroconductivity value,
d) a geographic location, and
e) optionally: a water source type
   and the output is a data indicative of whether fluoride content in the contacted water meeting one or more water quality criteria that pertain to fluoridation. By way of non-limiting example, the following can be ANN outputs:
a) a binary indication of fluoridation of the water source above a certain threshold, or within a certain range (delineated by a lower bound and a higher bound).
b) an estimated probability of fluoridation of the water source above a certain threshold, or within a certain range (delineated by a lower bound and a higher bound).
c) an estimated fluoridation value (e.g. in ppm, mg/L, etc.) of the water source

FIG. 1B illustrates an example water probe system for determination of water quality, in accordance with some embodiments of the presently disclosed subject matter. Probe body 110B can be composed of a water resistant material, can be handheld, and can be suitable for immersion in water. The probe can include handle 100B which can be attached to probe body 110B.

The probe can include water contact sensors 120B. Water contact sensors 120B can extend from probe body 110B, and can be suitable for immersion in a water source such as a spring or a reservoir. Examples of specific types of water contact sensors 120B are described below.

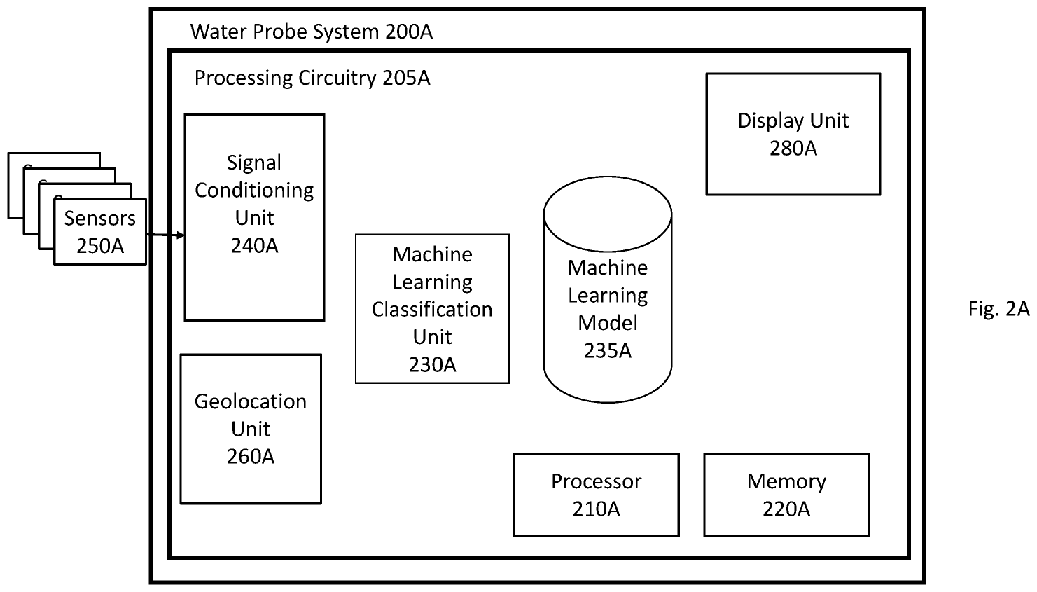

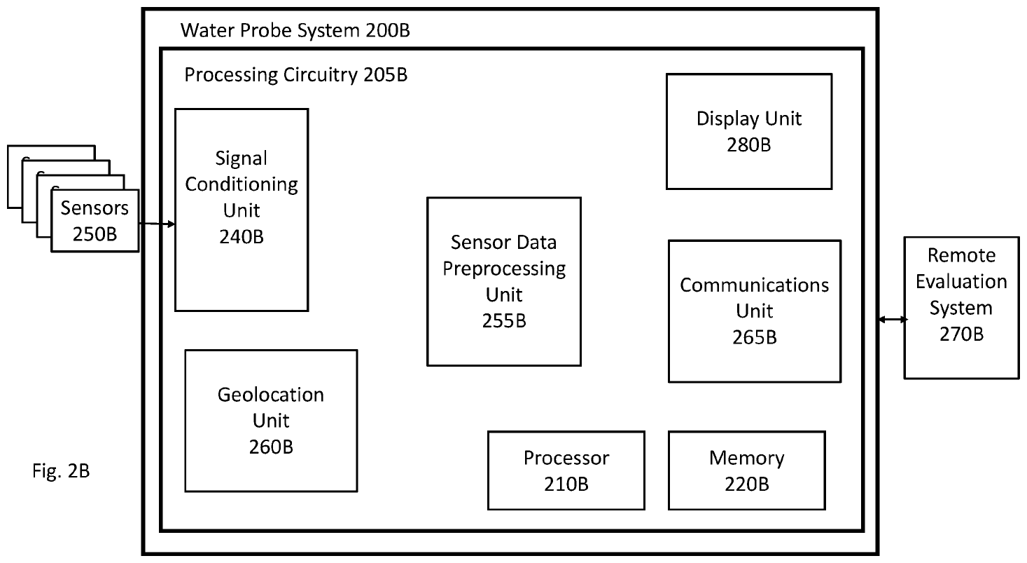

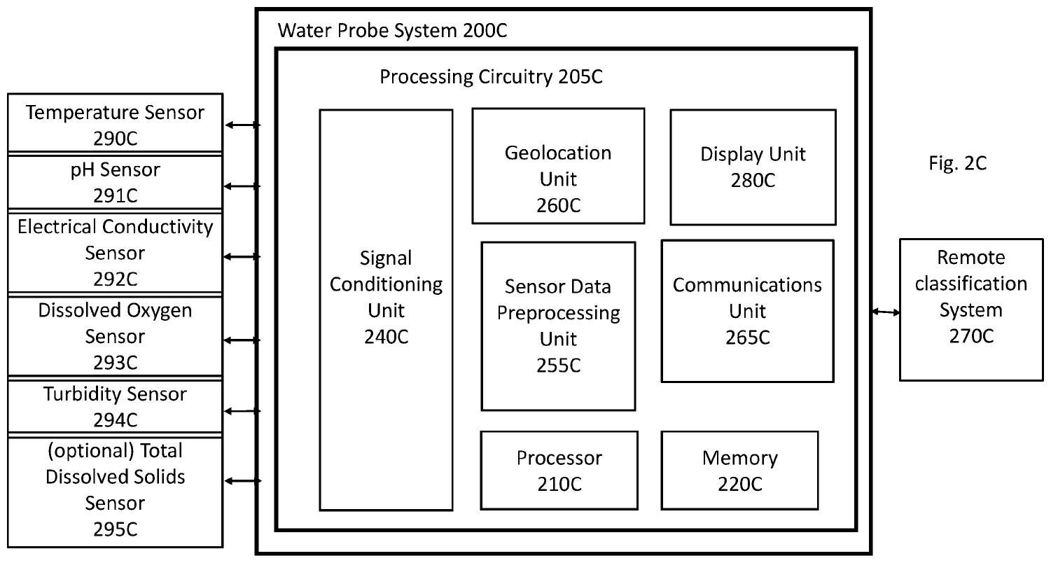

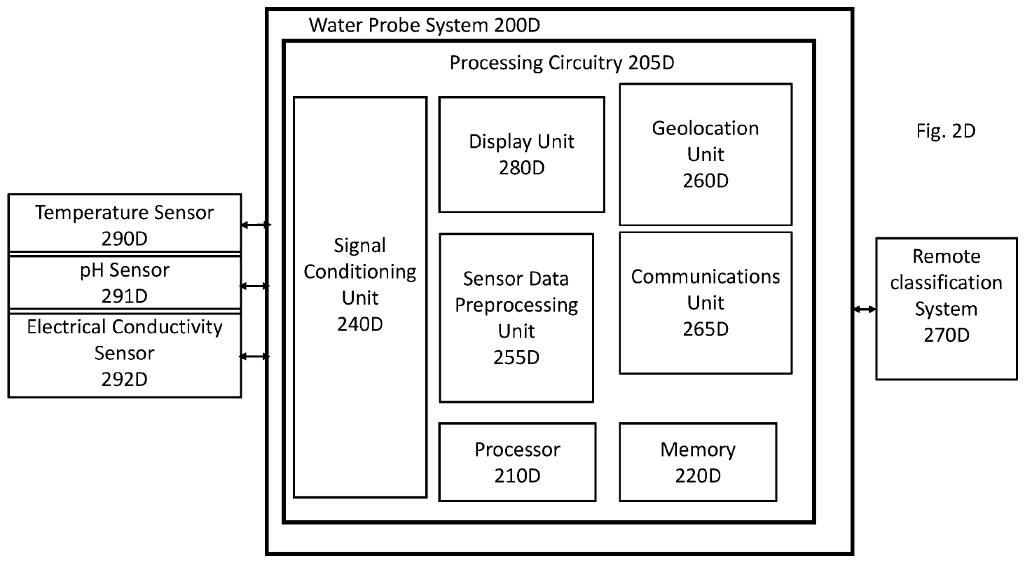

FIGS. 2A-2D are block diagrams of example water probe systems for determination of water quality, in accordance with some embodiments of the presently disclosed subject matter.

The water probe system 200A can include processing circuitry 205A.

Processor 210A can be a suitable hardware-based electronic device with data processing capabilities, such as, for example, a general purpose processor, digital signal processor (DSP), a specialized Application Specific Integrated Circuit (ASIC), one or more cores in a multicore processor, etc. Processor 210A can also consist, for example, of multiple processors, multiple ASICs, virtual processors, combinations thereof etc.

Memory 220C can be, for example, a suitable kind of volatile and/or non-volatile storage, and can include, for example, a single physical memory component or a plurality of physical memory components. Memory 220C can also include virtual memory. Memory 230 can be configured to, for example, store various data used in computation.

Processing circuitry 205C can be configured to execute several functional modules in accordance with computer-readable instructions implemented on a non-transitory computer-readable storage medium. Such functional modules are referred to hereinafter as comprised in the processing circuitry. These modules can include, for example, signal conditioning unit 240A, geolocation unit 260A, machine learning classification unit 230A, machine learning model 235A, and display unit 250A.

Sensors 250A can be operably connected to processing circuitry 205A. Sensors 250A can include contact elements which contact water and thereby enable detection/sensing of water properties. Signal conditioning unit 240A (for example) can then receive digital and/or analog signals from sensors 250A.

Sensors 250A can include, by way of non-limiting examples:
a) a pH sensor,
b) a temperature sensor,
c) an electroconductivity sensor,
d) a turbidity sensor,
e) a total dissolved solids sensor, and
f) a dissolved oxygen sensor

Signal conditioning unit 240A can transform raw output of analog sensors into a format (e.g. digital signals) usable by machine learning classification unit 230A or other components of processing circuitry 205A. Conversion by an Analogue-to-Digital Converter (ADC) can result in a digital value with varying resolution depending on the bit-length of the converter and settings used. For example: an 8-bit value can represent a “count” value between 0 and 1023 inclusive.

In some embodiments, the first transformation step is transforming the ADC raw results into the ADC voltage, which can be done by multiplying the raw ADC raw by a known constant. The next step can be converting the ADC voltage to a digital value indicative of the sensor's measurement e.g. by utilizing a calibration equation. For some sensors voltage (e.g. dissolved oxygen, conductivity, pH, and TDS), it can be further required to provide the temperature along with the ADC voltage).

Geolocation unit 260A can be a suitable kind of subsystem that ascertains the current geographic location of the probe. In some embodiments, geolocation unit 260A is global positioning system (GPS) that uses satellites to determine the current geographical location. In some embodiments, geolocation unit 260A is a user interface enabling manual entry of data indicative of the current location. The geographic location utilized can be provided at various levels of precision (e.g. longitude/latitude coordinates, a name of a geographic district, identifier corresponding to geographic district etc.)

Machine learning classification unit 230A can utilize sensor data and geolocation data (or data derivative of data and geolocation data), in conjunction with machine learning model 235A, to evaluate whether contacted water satisfies a particular water quality criterion. For example, machine learning classification unit 230A can perform machine learning classification using machine learning model 235A, thereby giving rise to a probability or other data indicative of whether the contacted water meets particular water quality criteria e.g. using machine learning based methods described above with reference to FIG. 1A.

Non-limiting examples of water quality criteria include: presence of fecal coliforms, of whether the fluoride level of the water meets a fluoridation threshold.

Optional display unit 250A can be any kind of user interface for displaying the results of water quality determination (e.g. a text window or other kind of screen).

The variant system shown in FIG. 2B utilizes a remote system to perform the evaluation (or classification) of the sensor data and geolocation data. By way of non-limiting example: sensor data preprocessing unit 255B can utilize communications unit 265B to transmit the sensor data and geolocation data (or data derivative of the sensor data and geolocation data) to remote evaluation system 270B.

Communications unit 265B can be a suitable type of wired or wireless transceiver (e.g. a cellular network station, bluetooth peer etc.)

Remote evaluation system 270B can be a suitable type of server (e.g. a physical server or cloud server). Remote evaluation system 270B can perform a method of determining whether the contacted water satisfies a water quality criterion e.g. using machine learning based methods described above with reference to FIG. 1A.

FIG. 2C illustrates an example variant system that can be suitable for detecting presence of fecal coliforms in water. Probe system 200C can include temperature sensor 290C, pH sensor 291C, electroconductivity sensor 292C, dissolved oxygen sensor 293C, turbidity sensor 294C, and (optionally) total dissolved solids sensor 295C.

Temperature sensor 290C can be a waterproof device that detects water temperature. The water's temperature can also impact other parameters such as pH, conductivity, dissolved oxygen, and total dissolved solids (TDS). Therefore, the temperature parameter can optionally be utilized in the calibration equation of these parameters to enable temperature compensation.

pH sensor 291C can measure the hydrogen ion concentration in a solution. The pH scale typically ranges from 1 to 14, with 7 representing a neutral pH. A pH below 7 indicates acidity, while values above 7 indicate alkalinity or basicity. The pH scale is logarithmic, meaning that each unit represents a tenfold difference in acidity or alkalinity.

Regarding electrical conductivity sensor 292C: conductivity is the reciprocal of resistance and is related to the material's ability to carry an electric current. In liquids, conductivity refers to measuring their ability to conduct electricity. In water, conductivity is an essential parameter used to assess water quality. It provides insights into the presence and concentration of dissolved ions and electrolytes in the water.

Dissolved oxygen sensor 293C can be a device used to measure the amount of dissolved oxygen in water, an important indicator of water quality. Such devices are widely utilized in various applications related to water quality assessment and monitoring as aquaculture, environmental monitoring, scientific research, and other areas where understanding and maintaining optimal dissolved oxygen levels in water are critical.

Regarding turbidity sensor 294C: suspended particles in water can be detected by measuring the light transmittance and scattering rate. Suspended particles, such as sediment, organic matter, or other solid particles, affect how light passes through the water. Monitoring turbidity helps to ensure compliance with water quality standards and provides valuable information about the clarity and overall health of the water body.

Regarding total dissolved solids sensor 295C: total dissolved solids (TDS) is a measurement that indicates the amount of soluble solids dissolved in water. TDS represents the total weight of all inorganic and organic substances in a given water volume. TDS indicates how many milligrams of soluble solids are dissolved in one liter of water.

FIG. 2D illustrates an example variant system that can be suitable for detecting whether fluoride level in water meets a fluoridation threshold. System 200D includes temperature sensor 290D, pH sensor 291D, electroconductivity sensor 292D. Remote classification system 270C can be accordingly trained to output data indicative of whether the contacted water meets one or more water quality criteria pertaining to fluoridation.

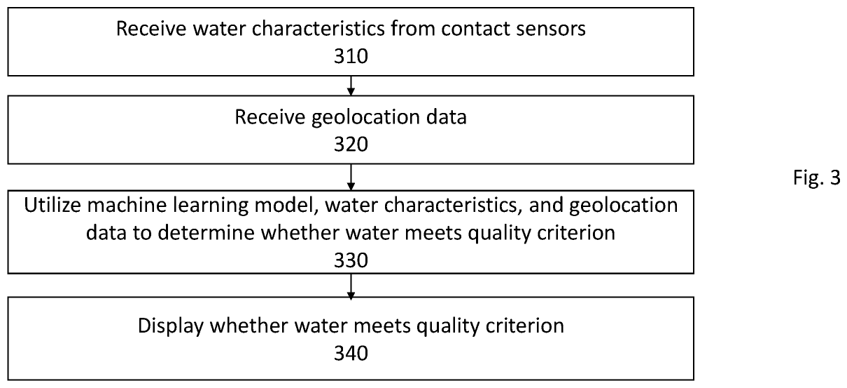

FIG. 3 is a flow diagram of an example method of determining whether contacted water meets a particular water quality criterion, in accordance with some embodiments of the presently disclosed subject matter.

Processing circuitry 205A (e.g. machine learning classification unit 255A) can receive 310 water characteristics (of contacted water) from contact sensors 250A (e.g. via signal conditioning unit 240A).

Processing circuitry 205A (e.g. machine learning classification unit 255A) can receive 320 geolocation data (e.g. a numeric value associated with a particular geographic area) from geolocation unit 260A.

Processing circuitry 205A (e.g. machine learning classification unit 255A) can next utilize 330 machine learning model 235A, received water characteristics, and received geolocation data to determine whether the contacted water meets a particular quality criterion (e.g using the method described above with reference to FIG. 1A).

Processing circuitry 205A (e.g. machine learning classification unit 255A) can then display 340 (e.g on display unit 250A) whether the contacted water meets the particular quality criterion.

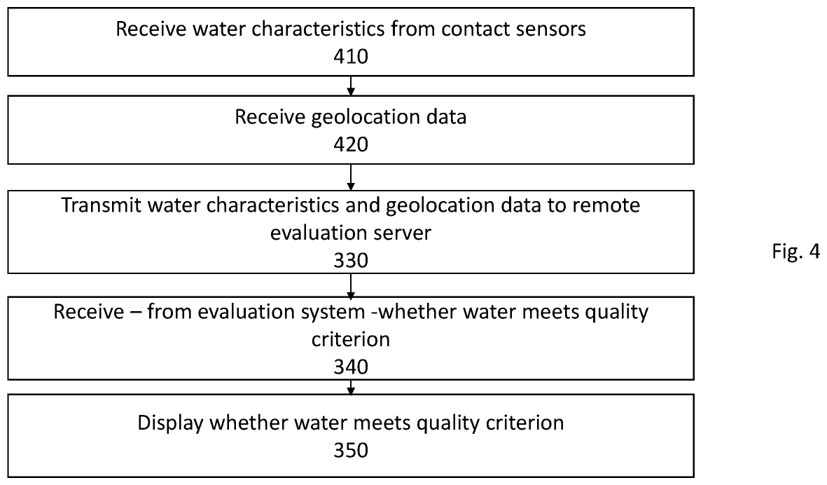

FIG. 4 is a flow diagram of another example method of determining whether contacted water meets a particular water quality criterion, in accordance with some embodiments of the presently disclosed subject matter.

Processing circuitry 205B (e.g. sensor data preprocessing unit 255B) can receive 410 water characteristics (of contacted water) from contact sensors 250B (e.g. via signal conditioning unit 240B).

Processing circuitry 205B (e.g. sensor data preprocessing unit 255B) can receive 420 geolocation data (e.g. a numeric value associated with a particular geographic area) from geolocation unit 260B.

Processing circuitry 205B (e.g. sensor data preprocessing unit 255B) can next transmit 430 water characteristics and geolocation data to remote evaluation system 270B (e.g. via communications unit 265B). Remote evaluation system 270B can then determine whether the contacted water meets the particular water quality criterion (e. g using the method described above with reference to FIG. 1A).

Processing circuitry 205A (e.g. sensor data preprocessing unit 255B) can then receive 440 (e.g from remote evaluation system 270B) whether the contacted water meets the particular water quality criterion.

Processing circuitry 205A (e.g. sensor data preprocessing unit 255B) can then display 450 (e.g on display unit 250A) whether the contacted water meets the particular water quality criterion.

It is to be understood that the invention is not limited in its application to the details set forth in the description contained herein or illustrated in the drawings. The invention is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the presently disclosed subject matter.

It will also be understood that the system according to the invention may be, at least partly, implemented on a suitably programmed computer. Likewise, the invention contemplates a computer program being readable by a computer for executing the method of the invention. The invention further contemplates a non-transitory computer-readable memory tangibly embodying a program of instructions executable by the computer for executing the method of the invention.

Those skilled in the art will readily appreciate that various modifications and changes can be applied to the embodiments of the invention as hereinbefore described without departing from its scope, defined in and by the appended claims.

1. A water quality probe system, the system comprising:
a) one or more water contact sensors;
b) a geolocation unit;
c) a processing circuitry (PC), operably connected to the one or more water contact sensors and to the geolocation unit, the PC being configurable to:
a. receive, from one or more of the water contact sensors, data indicative of one or more water characteristics sensed from contacted water;
b. receive, from the geolocation unit, data indicative of a current geographical location; and
c. determine data indicative of whether the contacted water satisfies one or more water quality criteria, utilizing, at least, the received sensed water characteristics and the current geographical location.

2. The system of claim 1, wherein at least one of the one or more water contact sensors is selected from of a list consisting of:
a. a pH sensor,
b. a temperature sensor,
c. an electroconductivity sensor,
d. a turbidity sensor,
e. a dissolved oxygen sensor, and
f. a total dissolved solids sensor.

3. The system of claim 1, wherein the geolocation unit is a global positioning system (GPS).

4. The system of claim 1, wherein the system is handheld.

5. The system of claim 1, wherein the PC is configured to perform a.-c.

6. The system of claim 5, wherein the PC is further configured to perform the determining by utilizing, at least, a trained machine learning model.

7. The system of claim 5, additionally comprising a communications link, and wherein the PC is further configured to perform the determining by receiving, from a remote server, data indicative of the whether the contacted water satisfies one or more water quality criteria,
the receiving from the remote server being at least partially responsive to the PC transmitting, at least, data derivative of at least part of the received sensed data and data derivative of the current geographical location to the remote server.

8. The system of claim 5, wherein the PC is further configured to:
d. present, on a user interface, an indication of whether the contacted water satisfies at least one criterion of the one or more water quality criteria.

9. The system of claim 6, wherein the one or more water sensors comprises:
a. a pH sensor,
b. a temperature sensor,
c. an electroconductivity sensor,
d. a turbidity sensor, and
e. a dissolved oxygen sensor; and
wherein the PC utilizes, as inputs to the trained machine learning model, at least:
a measured pH of the water source,
a temperature of the water source,
an electroconductivity of the water source,
a turbidity of the water source, and
a measured dissolved oxygen level of the water source; and
wherein at least one of the one or more water quality criteria determined by the trained machine learning model is:
whether a concentration of fecal coliforms in the contacted water lies between a given lower bound and a given upper bound.

10. The system of claim 9, wherein the one or more water sensors further comprises:
a total dissolved solids sensor, and
wherein the PC further utilizes a measured total dissolved solids of the water source as an input to the trained machine learning model.

11. The system of claim 7, wherein the one or more water sensors comprises:
a pH sensor,
b. a temperature sensor,
c. an electroconductivity sensor,
d. a turbidity sensor, and
e. a dissolved oxygen sensor; and
wherein the PC receives, at least, from the one or more water sensors, data indicative of:
a measured pH of the water source,
a temperature of the water source,
an electroconductivity of the water source,
a turbidity of the water source, and
a measured dissolved oxygen level of the water source; and
wherein at least one of the one or more water quality criteria is whether a concentration of fecal coliforms in the contacted water lies between a given lower bound and a given upper bound.

12. The system of claim 11, wherein the one or more water sensors further comprises:
a total dissolved solids sensor, and
wherein the PC further receives data indicative of a measured total dissolved solids of the water source.

13. The system of claim 6, wherein the one or more water contact sensors comprises:
a. a pH sensor,
b. a temperature sensor, and
c. an electroconductivity sensor; and
wherein the PC utilizes, as inputs to the trained machine learning model, at least:
a. a measured pH of the water source,
b. a temperature of the water source,
c. an electroconductivity of the water source; and
wherein one of the one or more water quality criteria determined by the trained machine learning model is whether fluoride content of the contacted water meets a fluoride content threshold.

14. The system of claim 7, wherein the one or more water contact sensors comprises:
a. a pH sensor,
b. a temperature sensor, and
c. an electroconductivity sensor; and
wherein the PC receives, at least, from the one or more water sensors, data indicative of:
a. a measured pH of the water source,
b. a temperature of the water source,
c. an electroconductivity of the water source; and
wherein one of the one or more water quality criteria received from the remote server is whether fluoride content of the contacted water meets a fluoride content threshold.

15. A computer program product comprising a non-transitory computer readable storage medium retaining program instructions, which, when read by a processing circuitry, cause the processing circuitry to perform a computerized method of determining whether contacted water satisfies one or more water quality criteria, the method comprising:
a) receiving, from one or more water contact sensors, data indicative of one or more water characteristics sensed from the contacted water;
b) receiving, from a geolocation unit, data indicative of a current geographical location; and
c) determining data indicative of whether the contacted water satisfies one or more water quality criteria, utilizing, at least, the received sensed water characteristics and the current geographical location.

16. A method of determining whether contacted water satisfies one or more water quality criteria, the method comprising:
a) receiving, from one or more water contact sensors, data indicative of one or more water characteristics sensed from the contacted water;
b) receiving, from a geolocation unit, data indicative of a current geographical location; and
c) determining data indicative of whether the contacted water satisfies one or more water quality criteria, utilizing, at least, the received sensed water characteristics and the current geographical location.

17. A system of determining whether a concentration of fecal coliforms in contacted water of a water source meets a fecal coliform concentration criterion, the system comprising a processing circuitry (PC) configured to:
a) receive, at least, data indicative of:
a geographic location of the water source,
a pH of the contacted water,
a temperature of the contacted water,
an electroconductivity of the contacted water,
a turbidity of the contacted water, and
a dissolved oxygen level of the contacted water; and
b) utilize, at least, the received data in conjunction with a machine learning model trained to determine data indicative of whether a concentration of fecal coliforms in contacted water of a water source meets the fecal coliform concentration criterion,
wherein the machine learning model was trained utilizing, at least, a set of training samples, wherein each training sample comprises:
data indicative of a geographic location of an origin of a respective water sample;
data indicative of a measured pH of the respective water sample,
data indicative of a temperature of the respective water sample,
data indicative of an electroconductivity of the respective water sample,
data indicative of a turbidity of the contacted water,
data indicative of a dissolved oxygen level of the contacted water, and
ground truth data indicative of whether a concentration of fecal coliforms in the sample meets the fecal coliform concentration criterion.

18. The system of claim 17, wherein the fecal coliform concentration criterion is whether the fecal coliform concentration lies between a given lower bound and a given upper bound.

19. The system of claim 17, wherein the geographic location comprises an identifier of a geographical region.

20. The system of claim 17, wherein the geographic location comprises a longitude and a latitude.

21. The system of claim 17, wherein the received data further comprises:
data indicative of an origin type of the water source;
and wherein each training sample further comprises:
data indicative of an origin type of the respective water sample.

22. A processing circuitry-based method of determining whether a concentration of fecal coliforms in contacted water of a water source meets a fecal coliform concentration criterion, the method comprising:
a) receiving, at least, data indicative of:
a geographic location of the water source,
a pH of the contacted water,
a temperature of the contacted water,
an electroconductivity of the contacted water,
a turbidity of the contacted water, and
a dissolved oxygen level of the contacted water; and
b) utilizing, at least, the received data in conjunction with a machine learning model trained to determine data indicative of whether a concentration of fecal coliforms in contacted water of a water source meets the fecal coliform concentration criterion,
wherein the machine learning model was trained utilizing, at least, a set of training samples, wherein each training sample comprises:
data indicative of a geographic location of an origin of a respective water sample;
data indicative of a measured pH of the respective water sample,
data indicative of a temperature of the respective water sample,
data indicative of an electroconductivity of the respective water sample,
data indicative of a turbidity of the contacted water,
data indicative of a dissolved oxygen level of the contacted water, and
ground truth data indicative of whether a concentration of fecal coliforms in the sample meets the fecal coliform concentration criterion.

23. A computer program product comprising a non-transitory computer readable storage medium retaining program instructions, which, when read by a processing circuitry, cause the processing circuitry to perform a computerized method of determining whether a concentration of fecal coliforms in contacted water of a water source meets a fecal coliform concentration criterion, the method comprising:
a) receiving, at least, data indicative of:
a geographic location of the water source,
a pH of the contacted water,
a temperature of the contacted water,
an electroconductivity of the contacted water,
a turbidity of the contacted water, and
a dissolved oxygen level of the contacted water; and
b) utilizing, at least, the received data in conjunction with a machine learning model trained to determine data indicative of whether a concentration of fecal coliforms in contacted water of a water source meets the fecal coliform concentration criterion, wherein the machine learning model was trained utilizing, at least, a set of training samples, wherein each training sample comprises:
data indicative of a geographic location of an origin of a respective water sample;
data indicative of a measured pH of the respective water sample,
data indicative of a temperature of the respective water sample,
data indicative of an electroconductivity of the respective water sample,
data indicative of a turbidity of the contacted water,
data indicative of a dissolved oxygen level of the contacted water, and
ground truth data indicative of whether a concentration of fecal coliforms in the sample meets the fecal coliform concentration criterion.

24. A system of determining whether fluoride content of contacted water of a water source meets a fluoride content criterion, the system comprising a processing circuitry (PC) configured to:
a) receive, at least, data indicative of:
a geographic location of the water source,
a pH of the contacted water,
a temperature of the contacted water,
an electroconductivity of the contacted water; and
b) utilize, at least, the received data in conjunction with a machine learning model trained to determine data indicative of whether fluoride content of contacted water meets the fluoride content criterion,
wherein the machine learning model was trained utilizing, at least, a set of training samples, wherein each training sample comprises:
data indicative of a geographic location of an origin of a respective water sample;
data indicative of a measured pH of the respective water sample,
data indicative of a temperature of the respective water sample,
data indicative of an electroconductivity of the respective water sample, and
ground truth data indicative of whether fluoride content of the water sample meets the fluoride content criterion.

25. A processing circuity-based method of determining whether fluoride content of contacted water of a water source meets a fluoride content criterion, the method comprising:
a) receiving, at least, data indicative of:
a geographic location of the water source,
a pH of the contacted water,
a temperature of the contacted water,
an electroconductivity of the contacted water; and
b) utilizing, at least, the received data in conjunction with a machine learning model trained to determine data indicative of whether fluoride content of contacted water meets the fluoride content criterion,
wherein the machine learning model was trained utilizing, at least, a set of training samples, wherein each training sample comprises:
data indicative of a geographic location of an origin of a respective water sample;
data indicative of a measured pH of the respective water sample,
data indicative of a temperature of the respective water sample,
data indicative of an electroconductivity of the respective water sample, and
ground truth data indicative of whether fluoride content of the water sample meets the fluoride content criterion.

26. A computer program product comprising a non-transitory computer readable storage medium retaining program instructions, which, when read by a processing circuitry, cause the processing circuitry to perform a computerized method of determining whether fluoride content of contacted water of a water source meets a fluoride content criterion, the method comprising:
a) receiving, at least, data indicative of:
a geographic location of the water source,
a pH of the contacted water,
a temperature of the contacted water,
an electroconductivity of the contacted water; and
b) utilizing, at least, the received data in conjunction with a machine learning model trained to determine data indicative of whether fluoride content of contacted water meets the fluoride content criterion,
wherein the machine learning model was trained utilizing, at least, a set of training samples, wherein each training sample comprises:
data indicative of a geographic location of an origin of a respective water sample;
data indicative of a measured pH of the respective water sample,
data indicative of a temperature of the respective water sample,
data indicative of an electroconductivity of the respective water sample, and
ground truth data indicative of whether fluoride content of the water sample meets the fluoride content criterion.
